(19) United States
(12) Patent Application Publication
(10) Pub. No.: US 2016/0095572 A1
(43) Pub. Date: Apr. 7, 2016

(54) System and Method for Non-Invasive Blood Pressure Measurement

(71) Applicant: Sperion Medical Devices, LLC, Austin, TX (US)

(72) Inventor: Derrick Aguren, Austin, TX (US)

(21) Appl. No.: 14/622,474

(22) Filed: Feb. 13, 2015

Related U.S. Application Data
(60) Provisional application No. 62/060,206, filed on Oct. 6, 2014.

(51) Int. Cl. A61B 8/04; A61B 8/08; A61B 8/00

(52) U.S. Cl. CPC A61B 8/04; A61B 8/4227; A61B 8/488; A61B 8/5276; A61B 8/5223

(57) ABSTRACT

A system and method for continuous real time measurement of blood pressure in a subject is presented. The system includes a transducer assembly (e.g., having ultrasound array elements) in a cuff applied to the subject's body. The system measures physical characteristics such as geometry, elasticity and strain in a blood vessel as well as other external physical parameters. Computer modeling and signal processing of measured signals are used during inflation and/or deflation of the cuff to iteratively estimate the blood pressure of the subject.

### RELATED APPLICATIONS

This application is related and claims the benefit of and priority to U.S. Provisional Application No. 62/060,206 entitled, “Non-Invasive Blood Pressure Measurement Device” filed on Oct. 6, 2014, which is hereby incorporated by reference.

### TECHNICAL FIELD

The present disclosure is directed to a system and method for estimating a continuous blood pressure waveform noninvasively using ultrasound and an automated cuff. It is in the class of hospital-quality continuous noninvasive blood pressure measurement (CNIBP) devices and is more accurate, faster, and more reliable for a wider range of patients than existing systems. An aspect of the present system and method is the use of shear wave elastography to directly measure elasticity. The present technique is extendable to other materials and shapes.

### BACKGROUND

The manual Riva-Rocci-Korotkoff (RRK) method is used for intermittent noninvasive blood pressure measurement (NIBP), which employs an inflatable cuff, a mercury or aneroid manometer, and auscultation (Reich (2011)). Though the method's clinical utility is proven, the accuracy of RRK in general suffers from human subjectivity, the need for trained users, and the need for regular calibration of aneroid devices (Reich (2011), de Greeff (2010)). Even when properly applied, RRK can be significantly inaccurate for patients with chronic hypertension, in shock, or with marked peripheral vasoconstriction (laizzo (2009)). Measurement time is on the order of minutes.

Other existing devices use an inflatable cuff. Oscillometric methods are employed in contemporary clinical settings. Strictly speaking only mean pressure is directly measured, while systolic and diastolic pressures are estimated by an empirically supported physical model (Drzewiecki (1994)). The need for regular calibration has historically contributed to systematic error (de Greeff (2010)). Even when properly calibrated, contemporary oscillometric devices can be inaccurate for low flow states (Reich (2011)), and have been shown to systematically overestimate systolic pressure for hypotensive patients with a significant epidemiological association with acute kidney injury and patient mortality in intensive care units (Lehman (2013)). The method can fail completely for weak pulses, for example with ill neonates (Lalan (2014)), and arrhythmia (laizzo (2009)). Measurement time is typically 30 seconds, and while “stat” back-to-back measurements can in principle be employed in emergency situations this usage has been associated with arm damage (Chung (2013)). The typical measurement rate is thus once every five minutes (Chung (2013)).

Modifications of the palpation method are used for patients in shock, low-flow states, and very young patients where Korotkoff sounds and oscillations are difficult to detect (laizzo (2009)). The Doppler method, the flush method, or pulse oximetry are used instead of finger palpation distal to a cuff to indicate the point of systolic pressure while deflating a cuff (laizzo (2009), Ribeiro (2011)). These methods are limited to systolic pressure and have similar accuracy and measurement rates as the RRK method.

The novel “pulse method” combines measurements from an automated upper arm cuff with a pressure sensor and a lower arm “pulse wave detector” to estimate intermittent brachial systolic and diastolic pressures (US 2013/0138001). A device based on this method has compared favorably to the oscillometric method (Xu (2014)).

In general, NIBP devices have high clinical utility but significant limitations. Most importantly their low sample rates categorically preclude detection of, and therefore clinical reaction to, acute blood pressure changing events occurring at a finer granularity than minutes.

Continuous intra-arterial (invasive) blood pressure measurement (IABP), also known as an arterial line, uses percutaneous cannulation with a catheter and an in-line electromechanical pressure transducer. IABP is the current gold standard of all blood pressure measurement methods and the comparative reference for all blood pressure devices (Chung (2013)). While any arterial site can be reached with the catheter via a variety of cannulation sites, radial artery cannulation is most common, providing a continuous waveform of radial or brachial arterial pressure (Chung (2013)). Use of an arterial line is indicated for monitoring of potentially rapid hemodynamic events such as during major surgery (Reich (2011)) or for intensive care unit (ICU) patients (Angus (2006)). For neonates, placement can be in the umbilical cord, and use is further indicated for “unstable conditions, such as lethargy, perfusion changes, or unstable respiratory conditions, clinical sepsis or proven sepsis, or severe cardiovascular disease” (Lalan (2014)). While more accurate than current NIBP methods, the arterial line is subject to the “end-pressure artifact”, biasing pressure measurements 2 to 10 mm Hg over the true pressure at the tip location (Reich (2011) pg. 47). The device is complex to use, contributing to systematic inaccuracy (Reich (2011) pg. 51). The risk of complications, possibly resulting in amputation (Reich (2011) pg. 48), or even death (Leslie (2013)), for this invasive procedure is offset by the seriousness of the patient's condition.

Continuous noninvasive blood pressure measurement (CNIBP), also known as continuous noninvasive arterial pressure measurement (CNAP), in principle combines the convenience and low risk of complications of NIBP methods with the measurement rate of IABP. CNIBP has been actively researched since the mid 19^{th} century (Chung (2013)).

Applanation tonometry uses a mechanical pressure sensor and a planar compressing surface to partially flatten the target artery against a nearby structure such as bone. This method directly measures sub-cycle continuous local pulse pressure (the difference between systolic and diastolic pressures) which is extrapolated to local diastolic, systolic, and mean pressures by various methods. The accuracy of estimated radial arterial pressures for a contemporary device has been described as “satisfactory” (Duek (2012)). Extrapolation of local pressures to continuous pressures at other sites can be improved by intermittent “calibrations” with a second NIBP device (Nelson (2010), Park (2014), Adji (2012), Salvi (2004)). The tonometric measurements have been shown to be achievable in laboratory conditions by Doppler ultrasound with similar application (Haluska (2007)). Further hemodynamic properties can be estimated by tonometric measurements at multiple sites, and with augmentation by electrocardiography (Adji (2012), Salvi (2004)). While the method cannot be directly applied to the brachial artery in general, radial artery applanation tonometry is considered the current gold standard for central (aortic) blood pressure estimation and is thus a potential substitute for brachial artery measurements (Park (2014)).

The Peñáz, a.k.a. volume clamp or vascular unloading, method uses an active cuff or pressure plate and continuous volume measurement (plethysmography) to measure the variation in applied pressure required to maintain constant local volume. Similar to applanation tonometry, the method directly measures continuous local pulse pressure which is then extrapolated to mean, diastolic, and systolic pressures by empirically-based estimates, “calibration” to a second NIBP device, and physiological models (Schattenkerk (2009)). Contemporary devices apply this method with finger cuffs for which recent validation studies show mixed results (Schattenkirk (2009), Garnier (2012), Hahn (2012)). Use of finger cuffs is further problematic for patients in any state with low peripheral perfusion, e.g. with peripheral vascular disease (Chung (2013)). Controlling for changes in muscle tone requires regular measurement interruptions (Bogert (2005)).

The pulse wave velocity (PWV), and the related pulse transit time (PTT), methods use the correlation of the arterial pulse pressure wave velocity to arterial stiffness, changes in artery cross-section geometry, empirical results, and optional calibrations with an NIBP cuff. The pulse transit time method directly estimates the pulse wave velocity from the measured travel time between two relatively distant arterial sites with known separation (Fung (2004)). The pulse wave dynamics, blood flow, and arterial geometry in contemporary methods are generally detected using various modes of ultrasound at two relatively close arterial sites or at a single site (Beulen (2011)). Validated commercial devices have not yet been produced from this method.

In general, the ambition of accurate, robust, CNIBP has not yet been achieved by contemporary methods and devices.

### BRIEF DESCRIPTION OF THE DRAWINGS

For a fuller understanding of the nature and advantages of the present invention, reference is made to the following detailed description of preferred embodiments and in connection with the accompanying drawings, in which:

FIG. 1 illustrates an exemplary embodiment of a device illustrating the principle of the invention applied to the brachial artery;

FIG. 2 illustrates an exemplary method according to aspects of the present disclosure; and

FIG. 3 illustrates an exemplary block diagram of a system according to aspects of the present disclosure.

### SUMMARY

The interior pressure of a pressurized vessel such as a blood-filled artery is estimated from a mechanical stress model of the container that is a function of current container geometry and known outer pressure. The stress model is parameterized by the viscoelastic properties of the container material and the geometry of its zero-stress state. If these parameters are not known a priori, then they are estimated from physical measurements of the container in one or more states of deformation. The quantity and range of measurements required is proportional to the container's elastic and geometric complexity. Vessels or containers with dynamic material properties, such as an artery with changing muscle tone, will have separate parameter solutions for each dynamic state. If the container is significantly pre-stressed, i.e. with internal stresses formed during its construction, then a direct measurement of inner pressure for some subset of deformations is required in order to account for this additional stress. For vessel dimensions constant under pressurization for the range of interest, incremental strain measurement can account for deformation in those dimensions attributable to changing external traction. Forces due to physical system motion can be compensated for directly via estimated densities, measured volumes, and measured acceleration.

The invention described here uses an external, pressure-sensing compressive mechanism to deform a pressurized vessel or container while taking measurements of its elastic properties, geometries, and, optionally, incremental strain. Note that if the target container has been stretched by external forces, or is significantly pre-stressed, zero transmural pressure is not the zero-stress state. The target vessel can be embedded in another medium to the extent that the embedding medium permits external compression, its geometry and viscoelastic properties are known or assumable, and it permits measurement of the target vessel properties of interest. The physical system deformation paths and measurements taken during motions due to external compression and coincident internal pressures are preferably equivalent or relatable to the physical system deformation paths and respective properties during “natural” motions due to internal pressures and ambient external pressures.

The invention described here samples a target vessel's phasic elasticity and geometry using shear wave elastography, an external compressive force, and known imaging methods in order to solve for the parameters of a mechanical stress model of that vessel or container. Deformation-causing inner pressure is then estimated on a continual basis, for known external pressures, from the container's current geometries for as long as those parameters remain valid. In general the invention applies to any system of vessels or containers for one or more embedded pre-stressed, anisotropic, non-linear viscoelastic, dynamically pressurized target vessels or containers.

The results obtained for a single site can be extended in a straight-forward manner to prediction of local pressures elsewhere in a connected pressurized system via supplemental measurements and/or empirical relation to measured values of one or more of geometries, elasticities, strain, and/or flow. Notably, while use of empirical relations may not produce as precise estimates as direct measurement, the estimates, and particularly the observation of relative changes in the estimates, can still be of high utility for regions where direct measurements are not possible, e.g. relative changes in pressure of the renal artery as an indication of stable perfusion pressure.

### DETAILED DESCRIPTION

A continuous local blood pressure waveform is produced by combining ultrasound-based artery cross-section acquisition and measurement, shear wave elastography, and strain imaging with an automated blood pressure cuff and a biomechanical model of the artery.

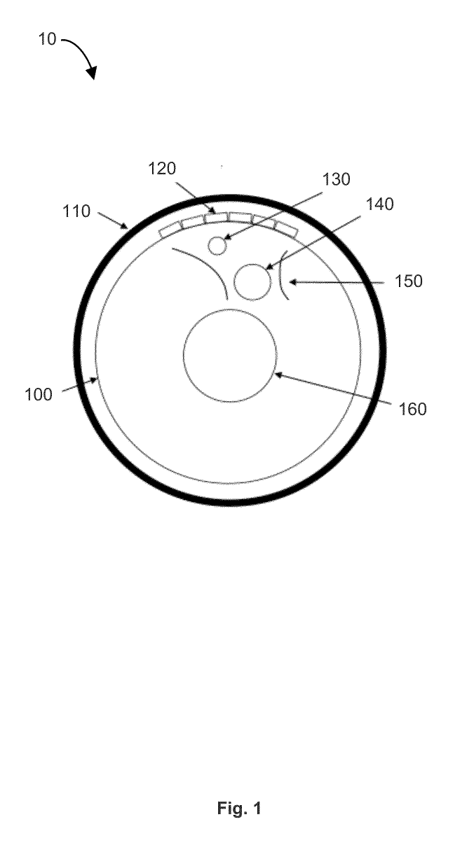

FIG. 1 illustrates an exemplary schematic perspective of a device 10 illustrating the principle of the invention applied to the brachial artery of a human upper arm utilizing an automated blood pressure cuff and a single ultrasonic rectangular transducer array to directly measure cross-section geometry, transverse and sagittal elasticity, and incremental axial strain. The arm surface is depicted in cross section by 100. The automated cuff 110 is placed about the arm 100. A transducer array 120 is disposed on or in cuff 110, e.g., on an inner surface of said cuff. A number of blood vessels may be present in the arm or limb of the patient. For example a target brachial artery 140 and a non-target vessel 130 are in a region subjected to ultrasonic energy envelope 150 of array 120. A bone 160 is also present, for example in the upper arm of the patient.

The system can interrogate, locate and/or track the target vessel 140 according to the following description.

It is to be noted that the shown example is only illustrative. Other applications in medicine, industry or other applications are possible. Those skilled in the art will understand that the present method and system can be applied to any fluid in a deformable vessel or fluid conduit consistent with the present teachings and models.

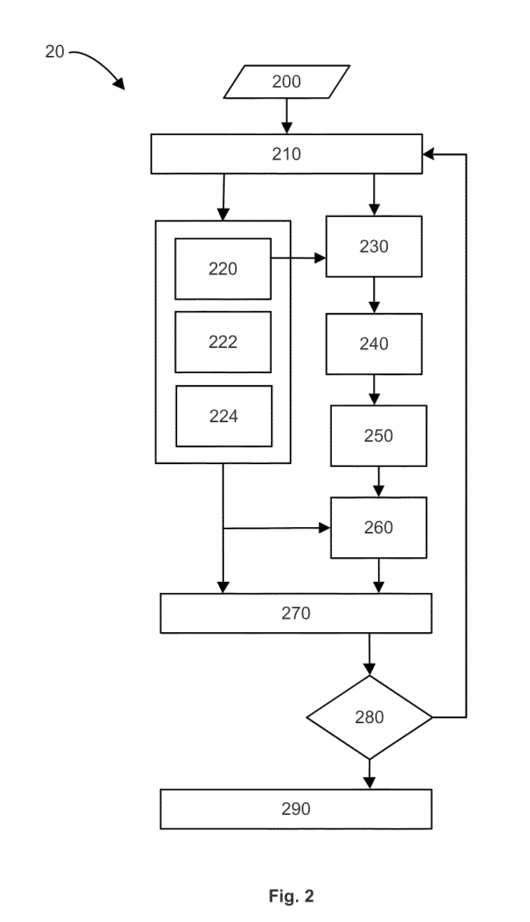

FIG. 2 illustrates steps of an exemplary method according to the present invention. Patient parameters are input as step 200 (or physical system if not in the context of a patient). The system and method acquire the target vessel (e.g., a blood vessel) in cross section at step 210, for example using the ultrasound cuff, adhesive patch or other distributed transducers. The system continuously measures cross-sectional geometry of the acquired target vessel at step 220. The system also continuously measures shear modulus and axial incremental strain at 222 and 224, respectively. An ultrasound array, e.g., a cuff is inflated to mechanically exercise the target vessel to or substantially to near a point of its collapse at 230. The zero transmural pressure geometry is estimated at 240. Then, the cuff is slowly deflated to a nominal cuff pressure at 250.

A biomechanical model parameter solution is obtained at 260 as described herein, and in the references cited herewith, which are hereby incorporated by reference. From the above, the system and method continuously (or substantially continuously) estimates blood pressure in the target vessel at 270 and detects change in muscle tone. The system determines if a new model solution is needed at 280. If YES, then the process repeats at step 230 supra. Also, the method and system displays the estimated blood pressure at 290, which is output to a display unit or other device as preferred.

Those skilled in the art will appreciate that alternate steps can be substituted for the exemplary steps in some cases based on a particular implementation. Also, that some of the steps may be performed in an order different than that shown. Finally, those skilled in the art will appreciate to include other steps not shown, and that some steps shown can be optional in particular embodiments without departing from the spirit of the invention.

In one aspect, the system and method accepts inputs including inputs relating to specifications or parameters regarding a subject. These parameters optimize artery acquisition and solving the biomechanical model by narrowing the respective search spaces. Examples of subject parameters that are input are the subject's species, age, approximate limb diameter, and artery type.

In another aspect, the system and method acquire target artery geometry, e.g., its cross-sections. One or more artery cross-sections are acquired and tracked using ultrasound imaging or other suitable means for generating such data. An embodiment employs ultrafast ultrasound B-mode imaging. Another embodiment utilizes ultrafast Doppler ultrasound imaging. Yet another embodiment combines more than one ultrasound imaging technique such as the foregoing techniques.

In yet other aspects, the system and method can continuously measure shear moduli of target vessels. Shear waves are induced in the proximal artery wall by applying an acoustic radiation force at regular intervals. The group, and optionally phase, velocities of the induced shear wave are then measured, optionally specific to directions of propagation. The co-directional shear moduli in time are then estimated from these velocities. An embodiment uses a supersonic shear wave imaging (SSI) method to estimate the absolute transverse shear moduli in time, further applying the same method in a circumferential direction to estimate the instantaneous sagittal shear moduli.

Still another aspect of the system and method continuously measures cross-sectional geometry of a target artery. Artery inner (lumen) and outer (media-adventitia or adventitia-perivascular tissue interface) radii are measured at regular intervals using B-mode ultrasound imaging and edge-detection methods. In some embodiments, the artery radius estimates can be improved using an incompressibility assumption. An embodiment uses ultrafast B-mode ultrasound imaging with real-time edge-detection.

Further aspects of the system and method continuously measure incremental axial strain in the target vessel. The length of an artery is generally constant under the influence of physiological pressure but can change significantly for physiological movement such as arm flexion. In an embodiment therefore, incremental axial (longitudinal) strain is measured ultrasonically at regular intervals using speckle-tracking methods. An embodiment uses ultrafast strain imaging for incremental artery longitudinal strain measurement.

Other aspects of the system and method inflate the cuff to artery collapse. An automated cuff is inflated until detection of artery collapse occurs, and pre-collapse geometries are measured by ultrasound and recorded. Artery collapse may be detected as a sudden significant change in cross section geometry, noting that collapse need not be total. Also, collapse identification can be augmented by a detected drastic change in local blood flow as measured by Doppler ultrasound.

Aspects of the system and method can also estimate zero transmural pressure geometry. The zero transmural pressure geometry is estimated from measured pre-collapse geometries, optionally corrected by empirical models.

Yet other aspects of the system and method slowly deflate the cuff to nominal cuff pressure. After estimating zero transmural pressure geometry, the cuff is slowly deflated at a configurable rate to allow shear moduli and associated geometry and incremental axial strain measurements to be made through a wide range of artery elastic responses from at, or near, zero transmural pressure to current systolic pressure and nominal cuff pressure. Deflation speed is a function of sample rates, the assumed maximum time of constant muscle tone, the minimum number of data points required for a sufficiently accurate blood pressure estimate, and clinical utility.

The system and method also solve biomechanical models using the above steps and measurements. Zero transmural pressure geometry measurements and co-incident shear moduli, cross-section geometry, and incremental axial strain measurements may be used as inputs to an optimization problem solving for the parameters of a non-linear system of parametric expressions of these or related values. If diastolic and systolic pressure estimates are available as a by-product of co-incident NIBP cuff measurements, these may optionally be used to calibrate the model as additional boundary conditions.

In other aspects, the system and method can continuously estimate blood pressure and detect change in muscle tone. Blood pressure is continually estimated from a parametric expression of artery inner pressure as a function of current geometry and incremental axial strain for the current model solution. Elasticity is continuously measured for current geometries and compared against predicted elasticity for the current model solution where a significant difference indicates a change in muscle tone.

A increase of blood pressure beyond the systolic pressure of the current model solution or a change in muscle tone may add significant error to the current model solution. These increases in blood pressure measurement uncertainty are optionally reported to the user. A new model solution is required if pre-defined error limits are exceeded or anticipated to be exceeded. Previous model solutions are cached, allowing blood pressure to be estimated from surface interpolation between previous solutions for the current measured elasticity and geometry instead of solving for new model parameters.

Other model solutions may be employed, e.g., in which a series of steps beginning with inflating the cuff to artery collapse (or substantially collapsing the artery by catastrophic spatial distortion thereof) and ending with a new model solution. Note that, since geometry measurements remain continuous during this process, if previous model solutions are available within pre-defined error tolerances, these estimates are reported to the user so that blood pressure measurements are uninterrupted.

In another aspect, the present system and method may display continuous blood pressure estimate. The blood pressure waveform and associated computed data such as systolic, diastolic, and mean arterial pressure per cardiac cycle are reported to the user through a configurable device external interface using known methods and technologies.

The method does not constrain transducer types, noting that transducers utilizing higher frequencies and lower signal-to-noise ratios improve measurement accuracy proportionally. The method does not constrain array configurations and sizes, noting that larger and shaped arrays may improve target acquisition, compensation for off-axis placement, penetration of thicker surrounding mediums, and access to complex target geometries. Arrays can be embedded in the cuff, disposed on an inner surface of the cuff, an outer surface of the cuff, or be kept as physically separate from the cuff but in geometric relation thereto.

In another embodiment, a blood pressure measurement device comprises a separate micro-machined ultrasonic transducer (MUT) patches affixed to the patient, e.g., on a self-adhesive patch. Acoustic coupling gels may be used between the transducer and body surface as necessary. Off-axis orientation of artery cross-sections to the normal of the transducer array may be compensated for by beam steering in some embodiments. Simultaneous measurements of shear moduli, geometry, and axial strain at multiple nearby sites within the effective region of a single cuff improves accuracy proportional to the number of additional sites. For the blood pressure measurement device this may entail multiple MUT patches. Measurements in time may be filtered and smoothed using digital signal processing methods as would be understood by those skilled in the art.

In general, shear wave inducement and wave velocity imaging can be accomplished by any known method appropriate to the target media, including, without limitation, a combination of acoustic radiation force and MRI or OCT for clinical applications, or seismic methods for geological systems.

It should be appreciated that the present system and method can be applied to a wider variety of applications than merely for estimating blood pressure in a blood vessel of a patient. For example, other situations where a fluid is contained in or flowing through a deformable vessel (pipe, conduit, hose, etc.) are also contemplated herein.

Referring to FIG. 2, the Transducer A/D Complex 230 may comprise sets of samplers, digital storage memories, and processing units per transducer, converting continuous analog transducer electronic state into a stream of digital samples. A central processing unit and memory may control the individual transducer configuration parameters, such as central frequencies and power output, and the dynamic temporal behavior of each transducer.

The Processor Complex contains electronic components, memories, and one or more CPUs dedicated to the configuration and execution of the top-level method, including cuff controller command sequences, edge-detection and radii estimation, zero-transmural pressure geometry estimation, speckle-tracking and incremental strain estimation, shear modulus estimation, biomechanical model solving, quantification of muscle tone state, error quantification, computation and formatting of results, and result forwarding.

It should be appreciated and will be understood by those skilled in the art that the processing circuits and processors described herein are sometimes general purpose processors, or can be custom hardware and microcontrollers and circuits, or ASIC (application specific integrated circuits). Generally, these things (processors) are adapted and connected to receive machine readable instructions, execute program instructions, etc. so as to operate on data, store, and otherwise manipulate electrical and electronic signals and information in the present context.

We now address various aspects and steps of the present method in further detail. It was mentioned that one or more steps of the method involve acquisition and/or tracking of the location of a vessel in the body of a patient, e.g., in a patient's limb. Artery acquisition can be semi-automatic, or automatic. In the semi-automatic process the artery is located by a trained operator and a displayed image such that the probe orientation is approximated normal to the artery. The location of the probe is replaced by the attached transducer arrays of the device described here and artery depth as reported by the probe is input into the device. In the automatic method, the device transducer arrays are attached by a trained operator in an approximate location and the artery is located in three dimensions by an iterative search for assumed geometric and ultrasonic response characteristics. Two dimensional transducer arrays compensate for incremental translation of the target. Conventional B-mode or an equivalent ultrafast imaging method are used, optionally augmented by Doppler flow imaging to further distinguish an artery from its environment based on blood flow.

Once the target artery is located, cross-sectional geometry is measured at regular intervals. For the geometry used in estimating shear moduli, the preferred geometry is the lumen radius and the radius of the tunica adventitia-perivascular tissue boundary. Due to a higher acoustic impedance, more precise ultrasonic measurements can be made of the tunica media-tunica adventitia boundary so these measurements are used preferentially, but without limitation, for solving the biomechanical model and predicting blood pressure. Using tunica media-tunica adventitia geometry will result in higher predicted intramural stresses than in reality. Geometry measurements may be extracted from longitudinal two dimensional images using a real-time implementation. Geometry measurements in time are preferentially filtered and smoothed using known methods.

Geometry measurements are taken preferentially for the same artery segment as the shear moduli estimates. Higher rates of geometry measurement can be achieved however by simultaneous measurements longitudinally or circumferentially offset from the site of acoustic radiation force application. Multiple nearby geometry measurements can further be averaged to improve co-incident measurement accuracy. Each additional site would require additional transducers, e.g., an additional 128 element linear array oriented longitudinally.

Transducer type and array shape are not limited here, but it is noted that geometry measurement accuracy improves with frequency and higher signal to noise ratio (SNR) may impacts the accuracy of the blood pressure measurement.

In other steps, the system and method may estimate a zero-transmural pressure geometry, or artery collapse due to an external band of pressure. Notably collapse pressure exceeds the zero transmural pressure condition, and collapse occurs rapidly after exceeding zero transmural pressure. Zero transmural pressure geometry is here therefore estimated from the geometry immediately preceding collapse as measured by ultrasound under increasing cuff pressure. Since the in vivo artery and perivascular tissue materials and geometry are not ideal, local buckling will occur for cuff pressures in the vicinity of zero-transmural pressure. Estimates of the zero transmural pressure condition are therefore optionally improved by an empirically-derived relationship between detected geometric eccentricities and the zero transmural pressure condition.

In yet other steps, the present method and system may estimate instantaneous shear moduli. A series of instantaneous shear moduli of an in vivo artery may be estimated by generating one or more shear waves in the arterial wall with a focused acoustic radiation force, measuring the phase velocities of the directionally dispersed waves, and fitting the dispersion curves to a family of a priori curves for homogeneous materials varying in the bulk shear wave speed for known geometry. The recovered bulk shear wave velocity is proportional to the respective instantaneous directional shear modulus. The estimation process may comprise two sequential phases: data acquisition, and postprocessing.

According to an exemplary embodiment, in the data acquisition phase, a linear transducer array, e.g., 8 MHz central frequency, 5-11 MHz bandwidth, 0.3 mm pitch (center-to-center distance), and a modified Aixplorer scanner from SuperSonic Imagine (Aix-en-Provence, France) which switches between conventional B-mode imaging, acoustic radiation force sequence (push sequence), and an ultrafast imaging sequence. Coherent compounding of tilted plane waves may be used to increase resolution and contrast of the ultrafast imaging sequence. Also, multiple elasticity maps may be constructed from different steered shear-plane waves can be averaged to improve accuracy (shear compounding).

According to one or more aspects, a push sequence and ultrafast imaging sequence are employed and may be applied in three successive steps.

First, using a reference step. A reference ultrafast image is taken for correlation of displacements to current geometry. Artery segment thickness (including the adventitia) and lumen radius at the time of the shear wave may be measured with ultrasound using an unspecified method (presumably B-mode). Vessel geometry may be derived from the reference image, preferably using compound coherent plane imaging as described above.

Second, a series of pushing beams are transmitted in time at successive depths to achieve source activation. For example, a series of five beams at a 5 mm separation for a 40 mm focal target (at 15, 20, 25, 30, 35 mm), with a mean focal depth at the center of the proximal arterial wall may be used. Each pushing beam may have a burst duration of 100 microseconds. The pushing beams may be positioned manually by an operator via ultrafast images. This may also be done automatically using an iterative optimization method and beam steering.

Third, the ultrafast sequence may be triggered automatically when the pushing beams and arterial wall are aligned. In an aspect, electrocardiogram measurements may be taken during the data acquisition process for association of the estimated moduli in time with the cardiac cycle, but this is not needed for shear modulus estimation. The electrocardiogram measurements may be taken during the data acquisition process for association of the estimated moduli in time with the cardiac cycle, but again, this is not needed for shear modulus estimation.

The present system and method may employ postprocessing of data. The following are exemplary but not limiting aspects of postprocessing steps which can be applied as needed in some embodiments.

In one example, for an ultrafast plane wave insonification, the ultrasonic backscattered echoes (radiofrequency data) are sampled and stored in per-transducer memories are transferred to a central memory and beamformed to create a stack of IQ-demodulated complex (I+Qi) 2D (line x depth) images with a demodulation frequency equal to the transmit frequency (no down mixing for attenuation).

In another example, frame-to-frame axial velocity estimation (in line x depth) is performed using an IQ cross-correlation (with the reference image) algorithm. This may be applied aside from Doppler imaging as frequencies may be unchanged.

In yet another example, frames may be compiled into an animated sequence or “movie” of axial velocity in depth, line number, and frame number. In a particular example having a typical 50 images at 8000 images per second.

Still another example applies axial wall translation and is controlled for using a speckle tracking algorithm. Other examples employ an axial velocity in time matrix for the upper wall (proximal to the transducer array) which may be derived from the movie or animated sequence mentioned above.

In other examples, pulsatile motion is corrected by removing the time average of particle velocity for each line of the image.

In further examples, a 2-D Fourier transform is applied resulting in a frequency x phase velocity x transform amplitude.

In still other examples, a frequency x phase velocity dispersion curve is extracted at the maximal Fourier transform amplitude for each frequency.

In yet other examples, the measured dispersion curve may be the dependent variable for a priori dispersion curves in bulk shear wave speed and geometry, for the measured geometry, such that bulk shear wave speed is recovered as an inverse problem.

For a homogenous free plate (in vacuum) and A₀ mode guided Lamb waves, the dispersion equation for low frequencies is approximated by:

$${v}_{{A}_{0}} = \sqrt{\frac{ω {hc}_{T}}{\sqrt{3}}}$$

such that shear bulk velocity (plate transverse wave bulk velocity) c_{T} is recovered from known phase velocities v_{A}_{₀}, frequencies w, and geometry (thickness h).

For a homogenous plate immersed in a nonviscous liquid or solid, the dispersion equation includes a loss of energy due to leakage.

The dispersion equation for a homogenous medium with mixed boundary conditions (semi-infinite liquid/solid layer/semi-infinite solid) is not tractable analytically, and so is solved numerically or empirically.

Dispersion in the heterogeneous artery is modeled incrementally and as a homogenous plate in the longitudinal direction for assumed boundary conditions and instantaneous geometry.

Assuming a homogenous medium in free space, the isotropic shear modulus μ and Young's modulus E are directly proportional to the recovered bulk shear wave speed for assumed density ρ:

μ=ρc_{T}², E≈3μ.

Treating the shear modulus as directional (i.e. specific to shear motion in the transverse plane of the artery), the method is further applied to estimate the instantaneous sagittal shear modulus for shear waves propagating in the circumferential direction using one or more additional transducer arrays. Either the circumferential phase velocities of the weak circumferential shear wave of the longitudinally-oriented shear-plane wave are measured by the circumferentially oriented transducers, or a separate circumferentially-oriented set of shear-plane waves are induced by the circumferentially oriented transducers. Generally shear-plane waves can be generated and their phase velocities measured in any orientation, resulting in estimations of combinations of shear moduli. Multiple shear wave directions can be applied to the same segment in alternation, or nearby segments simultaneously assuming equivalent geometric and material properties.

The present system and method is also concerned with biomechanical models of the objects under testing. In this respect, a validated, three-dimensional, finite deformation, non-linear, constitutive stress model may be derived from an assumed strain-energy function using the principles of continuum mechanics applied to arteries. The vessel of interest, e.g., an artery, may be modeled as an incompressible, pre-stressed, thick-walled, anisotropic, hyperelastic cylinder comprised of four fiber families embedded in an isotropic medium. The uniform continuum of fiber families in the circumferential-axial planes are phenomenological models of the directional, non-linear elastic behavior of similarly oriented artery collagen and superpositioned circumferentially-oriented smooth muscle, while the isotropic medium represents the non-directional (by assumption), semi-linear elastic behavior of the elastin and intracellular matrix constituents. Strain-energy is expressed in a cylindrical coordinate system as a function W(I_{C}) of the principal strain invariants I₁ and I₂ of the right Cauchy-Green deformation tensor C=F^{T}·F for deformation tensor F and additional “fiber” strain invariants I₄⁽ⁱ⁾, I₅⁽ⁱ⁾, I₆⁽ⁱ⁾ and I₇⁽ⁱ⁾ for unit vectors U⁽ⁱ⁾=(U_{R}⁽ⁱ⁾, U_{Θ}⁽ⁱ⁾, U_{Z}⁽ⁱ⁾), V⁽ⁱ⁾=(V_{R}⁽ⁱ⁾, V_{Θ}⁽ⁱ⁾, V_{Z}⁽ⁱ⁾) in the reference (initial, zero stress) configuration, and vectors u⁽ⁱ⁾=FU⁽ⁱ⁾, v⁽ⁱ⁾=FV⁽ⁱ⁾ in the current (deformed) state (i=1,2):

$$W ( {I}_{C} ) = a ( {I}_{1} - 3 ) + b ( {I}_{2} - 3 ) + {\sum }_{i = 1}^{2} {\sum }_{j = 4}^{7} \frac{{k}_{i}^{( j )}}{2 {\overline{k}}_{i}^{( j )}} \{ exp [ {{\overline{k}}_{i}^{( j )} ( {I}_{j}^{( j )} - 1 )}^{2} ] - 1 \}$$

where (a, b) and kᵢ^{(j)} are “stress-like”, and k̅ᵢ^{(j)} are dimensionless, material parameters. Cauchy stress σ=J⁻¹F·S·F^{T} for second Piola-Kirchhoff stress tensor S=2∂W(C)/∂C, where J=det F=1 by the incompressibility assumption. This transforms W(I_{C}) into the three-dimensional Cauchy stress function σ(r, t), here neglecting the contributions of I₂, I₅⁽ⁱ⁾, and I₇⁽ⁱ⁾ per simplifying assumptions, and where incompressibility is enforced by a Lagrange multiplier p:

$$σ ( r , t ) = p 1 + 2 {W}_{1} B + 2 {\sum }_{ i = 1}^{2} ( {W}_{4}^{( i )} {u}^{( i )} ⊗ {u}^{( i )} + {W}_{6}^{( i )} {v}^{( i )} ⊗ {v}^{( i )} )$$

where W₁=∂W/∂I₁ and Wₖ⁽ⁱ⁾=∂W/∂Iₖ⁽ⁱ⁾ (k=4,6), and the 1 in the first term represents the identity tensor.

Using simplifying assumptions results in the following set of material and geometric parameters:

Material:
a Isotropic (Neo-Hookean) contribution
kₐ, k̅ₐ Diagonal and axial (longitudinal) fiber family contribution
k_{b}, k̅_{b} Circumferential fiber family contribution

Diagonal fiber families initial angle with respect to the
α

longitudinal axis

Geometric:
Rₒ Open sector outer radius in the reference configuration
Θ₀ Open sector opening angle in the reference configuration
λ Axial stretch in the current configuration

We note that while these parameters are motivated by specific structural and micro-structural features they are not mathematically independent for this model. Multiple parameter solutions can describe the behavior of the same object in its current geometries. Parameters may also vary with respect to the range of deformations they describe. By extension, the interpretation of a specific derived response of interest, e.g. circumferential stress, depends on the data set for which the model is solved.

The expression for inner (blood) pressure Pᵢ(t), and similarly transmural pressure Pₜ(t)=Pᵢ(t)−Pₒ(t) for outer pressure Pₒ(t), is derived from the equations of motion in the absence of body forces, here neglecting the inertial contribution per a simplifying assumption:

$${P}_{i} ( t ) = {P}_{o} ( t ) + {\int }_{{r}_{i} ( t )}^{{r}_{o} ( t )} \frac{{σ}_{θθ} ( r , t ) - {σ}_{rr} ( r , t )}{r} d r$$

where rᵢ(t) and rₒ(t) are the artery current inner and outer radius, respectively.

In an aspect, the present method and system may account for varying axial (longitudinal) length in time due to physiological movement by including an incremental strain modifier for the constant in vivo axial stretch parameter:

$$λ \to λ ( t ) ≡ λ ( {ɛ}_{inc}^{*} ( t ) ) = λ {\prod }_{i = 1}^{n} ( {ɛ}_{inc} ( {t}_{i} ) + 1 )$$

for incremental axial strains ε_{inc}*(t)≡ε_{inc}(tᵢ)=(l(tᵢ)−l(tᵢ₋₁))/l(tᵢ₋₁) for some artery segment with axial lengths l at two instances in time for i=1 . . . n measurements with tₙ at or near current time t.

Further extending the above results, expressions for spatial elasticity tensor components sagittal shear modulus μ_{rθ}(r, t) and transverse shear modulus μ_{rz}(r,t) may be derived using a finite-differences numerical approximation:

$$\begin{matrix}{μ}_{r θ} ( r , t ) = {J}^{- 1} {F}_{rR} {F}_{θΘ} {F}_{rR} {F}_{θΘ} 4 \frac{{\partial }^{2} W}{\partial {C}_{R Θ} \partial {C}_{R Θ}} \\ = \frac{2 a ( \frac{{π}^{2} ε {r}^{2}}{{σ}_{1}} + \frac{{Θ}_{0}^{2} {εσ}_{6}}{2 {π}^{2} {λ ( t )}^{2} {r}^{2}} )}{ε} + \frac{{π}^{2} {k}_{b} {r}^{2} exp ( {{\overline{k}}_{b} ( {σ}_{4} + {σ}_{2} - 1 )}^{2} ) ( {σ}_{3} + \frac{{σ}_{5}}{{σ}_{1}} - 2 )}{{σ}_{1}} \\ \frac{{π}^{2} {k}_{a} {r}^{2} exp ( {{\overline{k}}_{a} ( {sin ( α )}^{2} ( {σ}_{4} + {σ}_{2} ) + {λ ( t )}^{2} {cos ( α )}^{2} - 1 )}^{2} ) {sin ( α )}^{2} ( {sin ( α )}^{2} ( {σ}_{3} + \frac{{σ}_{5}}{{σ}_{1}} ) + 2 {λ ( t )}^{2} {cos ( α )}^{2} - 2 )}{{Θ}_{0}^{2} {σ}_{6}}\end{matrix}$$

$$where$$

$${σ}_{1} = 2 {Θ}_{0}^{2} {σ}_{6}$$

$${σ}_{2} = \frac{{σ}_{5}}{4 {Θ}_{0}^{2} {σ}_{6}}$$

$${σ}_{3} = \frac{2 {π}^{2} {r}^{2}}{{Θ}_{0}^{2} {σ}_{6}}$$

$${σ}_{4} = \frac{{π}^{2} {r}^{2}}{{Θ}_{0}^{2} {σ}_{6}}$$

$${σ}_{5} = {π}^{2} {ε}^{2} {r}^{2}$$

$${σ}_{6} = {R}_{o}^{2} + \frac{πλ ( t ) ( {r}^{2} - {r}_{o}^{2} )}{{Θ}_{0}}$$

$$and$$

$$\begin{matrix}{μ}_{rz} ( r , t ) = {J}^{- 1} {F}_{rR} {F}_{zZ} {F}_{rR} {F}_{zZ} 4 \frac{{\partial }^{2} W}{\partial {C}_{RZ} \partial {C}_{RZ}} \\ = \frac{2 a ( \frac{{ελ ( t )}^{2}}{2} + \frac{{Θ}_{0}^{2} {εσ}_{1}}{2 {π}^{2} {λ ( t )}^{2} {r}^{2}} )}{ε} + \frac{{k}_{a} {λ ( t )}^{2} exp ( {{\overline{k}}_{a} ( \frac{{σ}_{3}}{4} + {λ ( t )}^{2} - 1 )}^{2} ) ( \frac{{σ}_{3}}{2} + 2 {λ ( t )}^{2} - 2 )}{2} \\ {k}_{a} {λ ( t )}^{2} exp ( {{\overline{k}}_{a} ( {cos ( α )}^{2} {σ}_{2} + \frac{{π}^{2} {r}^{2} {sin ( α )}^{2}}{{Θ}_{0}^{2} {σ}_{1}} - 1 )}^{2} ) {cos ( α )}^{2} ( 2 {cos ( α )}^{2} {σ}_{2} + \frac{2 {π}^{2} {r}^{2} {sin ( α )}^{2}}{{Θ}_{0}^{2} {σ}_{1}} - 2 )\end{matrix}$$

$$where$$

$${σ}_{1} = {R}_{o}^{2} + \frac{πλ ( t ) ( {r}^{2} - {r}_{o}^{2} )}{{Θ}_{0}}$$

$${σ}_{2} = \frac{{σ}_{3}}{4} + {λ ( t )}^{2}$$

$${σ}_{3} = {ε}^{2} {λ ( t )}^{2}$$

with respective means in radius r

$$\overline{μ} ( t ) = \frac{1}{{r}_{o} ( t ) - {r}_{i} ( t )} {\int }_{{r}_{i} ( t )}^{{r}_{o} ( t )} μ ( r , t ) d r$$

for some small perturbation ε, e.g. 10⁻⁸, keeping J=1.

To solve for the model parameters, a system f of parameterized equations in geometry and incremental axial strain is created for weighted zero transmural pressure expressions ξP₀^{(j)}(rᵢ⁰(tⱼ), rₒ⁰(tⱼ), ε_{inc}*(tⱼ)) in measured zero transmural pressure inner and outer radii, rᵢ⁰, rₒ⁰, and related incremental strains experienced during the measurement process for j=1 . . . n measurements, and mean shear modulus expressions μ̅_{rθ}^{(k)}(rᵢ(tₖ), rₒ(tₖ), ε_{inc}*(tₖ)), μ̅_{rz}^{(k)}(rᵢ(tₖ), rₒ(tₖ), ε_{inc}*(tₖ)) in measurements rᵢ(tₖ), rₒ(tₖ), ε_{inc}*(tₖ) and estimates \overline{\tilde{μ}}_{rθ}^{(k)}(tₖ), \overline{\tilde{μ}}_{rz}^{(k)}(tₖ) for k=1 . . . m measurements and coincident estimates:

$$f = [ \begin{matrix}ξ {P}_{0}^{( 1 )} \\ ⋮ \\ ξ {P}_{0}^{( n )} \\ {\overline{μ}}_{r θ}^{( 1 )} - {\tilde{\overline{μ}}}_{r θ}^{( 1 )} \\ ⋮ \\ {\overline{μ}}_{r θ}^{( m )} - {\tilde{\overline{μ}}}_{r θ}^{( m )} \\ {\overline{μ}}_{rz}^{( 1 )} - {\tilde{\overline{μ}}}_{rz}^{( 1 )} \\ ⋮ \\ {\overline{μ}}_{rz}^{( m )} - {\tilde{\overline{μ}}}_{rz}^{( m )}\end{matrix} ]$$

where weight ξ is pre-determined as a function of m and n, e.g. ξ=2m/10n The parameters of this system of non-linear equations are solved, without limitation, by minimizing the sum of squares of the elements of f using the trust-region-reflective non-linear least squares optimization method with empirically pre-determined parameter bounds and initial values. If an optimization instance minimizes poorly, in terms of a pre-determined tolerance, or fails altogether, then new initial values are iteratively chosen and tried until a solution meets pre-determined error tolerances. Here, without limitation, new initial values may be chosen via pseudo-random selection from a (bounded) pre-determined Gaussian distribution about the mean of the pre-determined parameter bounds.

Current estimated blood pressure \tilde{P}ᵢ(t) is then a function of the solved (constant) material and geometric parameters, current cross-section geometry (rᵢ(t), rₒ(t)),a series of incremental axial strain measurements ε_{inc}*(t) with respect to current time t, and known or assumed current outer pressure Pₒ(t):

\tilde{P}ᵢ(t)=Pᵢ(a, kₐ, k̅ₐ, k_{b}, k̅_{b}, α, Rₒ, Θ₀, λ, rᵢ(t), rₒ(t), ε_{inc}*(t), Pₒ(t))

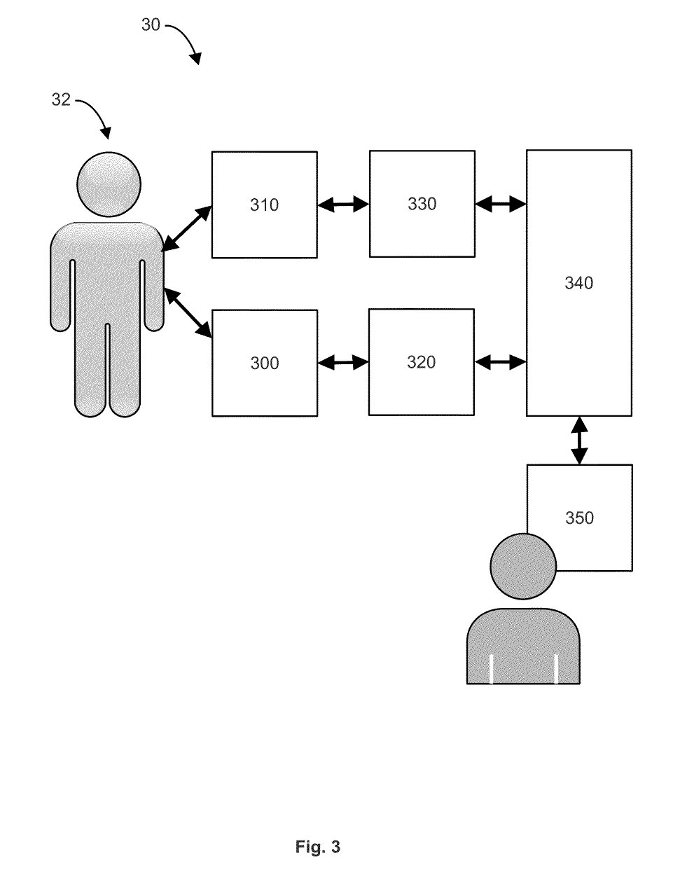

FIG. 3 illustrates a representative system 30 according to the present disclosure. A patient 32 has a limb containing a blood vessel the pressure in which we wish to determine (or estimate). The system 30 includes an ultrasound transducer complex 310, an ultrasound transducer analog to digital conversion (ND) complex 330, a processor complex 340, a cuff controller 320 to control the inflation, deflation and other aspects of the cuff 300. An operator or practitioner 34 controls the overall operation of system 30 through user interface (U/I) 350 and receives information (e.g., blood pressure) therethrough.

As mentioned above, the present system and method can be applied to a wider variety of problems than merely estimation of blood pressure in a blood vessel. Rather, it is intended that the present discussion be understood to extend to other fluids residing or flowing in other types of vessels, containers and deformable conduits.

The present invention should therefore not be considered limited to the particular embodiments described above. Various modifications, equivalent processes, as well as numerous structures to which the present invention may be applicable, will be readily apparent to those skilled in the art to which the present invention is directed upon review of the present disclosure.

What is claimed is:

1. A system for non-invasive and real-time blood pressure measurement, comprising:
a cuff configured and dimensioned to generally surround a limb of a patient, said cuff having mating ends to be mechanically secured about said limb;
said cuff further having a plurality of ultrasound transducers disposed on or with respect to said cuff, and configured and placed to transmit and/or receive ultrasound energy into and/or out of said limb;
a transducer driving circuit electrically coupled to said ultrasound transducers so as to drive each transmitting transducer with an electrical driving signal;
a processor electrically coupled to said ultrasound transducers so as to receive an electrical response signal from each receiving transducer;
a digital storage unit that stores data and program instructions allowing control of said electrical driving signals and processing said electrical response signals;
said processor further having circuitry to receive said electrical response signals and to determine at least geometric data regarding a blood vessel in said limb therefrom and to determine an axial strain in said blood vessel as well as to execute said stored program instructions and to process said stored data, including processing said program instructions, geometric data and axial strain to estimate a blood pressure within said blood vessel;
said processor further coupled to an output unit that conveys an output representative of said blood pressure parameter.

2. The system of claim 1, further comprising an accelerometer that senses an acceleration in one or more directions, said processor incorporating said acceleration in one or more directions in estimating said blood pressure.

3. The system of claim 1, further comprising an ambient pressure sensor that senses a pressure of an atmosphere or environment surrounding said limb.

4. A method for non-invasive real-time measurement of a variable fluid pressure in a deformable vessel having deformable walls that deform responsive to transmural forces thereon including a transmural pressure defined by an internal pressure within said vessel and an external pressure outside said vessel, the method comprising:
mechanically exercising said vessel through a plurality of phases of its geometry by controllably deforming said vessel walls during exercising of said vessel;
measuring an elasticity of said vessel while mechanically exercising said vessel;
measuring a geometry of said vessel while mechanically exercising said vessel;
measuring an incremental strain in said vessel while mechanically exercising said vessel, and calculating an absolute strain therefrom;
estimating at least one property of said vessel using said measured elasticity, geometry, incremental strain and a physical model of said vessel;
validating one or more parameter solutions used in said physical model of said vessel that includes said estimated at least one material property; and
estimating an interior pressure in said vessel using said physical model and a current measurement of said vessel's geometry, axial strain and external pressure.

5. The method of claim 4, said vessel comprising a blood vessel and said transmural pressure defined by an external pressure acting on an external surface of said blood vessel and an internal blood pressure within said blood vessel.

6. The method of claim 5, estimating said interior pressure further providing a blood pressure measurement in said blood vessel.

7. The method of claim 4, further comprising applying an array of transducers outside said vessel and using said array of transducers to make said geometry measurement.

8. The method of claim 7, applying said array of transducers comprising applying said array of transducers to a cuff surrounding a patient's limb so as to measure said geometry of a blood vessel in said patient's limb.

9. The method of claim 4, said at least one property comprising at least one material property.

10. The method of claim 4, said at least one property comprising at least one geometric property.

11. The method of claim 4, further comprising detecting changes in smooth muscle tone in said patient's limb and using said changes in smooth muscle tone in the step of validating said physical model.

12. The method of claim 4, further comprising exercising said vessel to achieve a substantial collapse of said vessel.

13. The method of claim 4, further comprising acquiring and tracking a position of said vessel within a portion of an anatomy containing said vessel.

14. The method of claim 13, said acquiring and tracking comprising using a search algorithm to acquire the vessel and to further track its location within a region of interest.

15. A system for non-invasive and real-time pressure measurement of a fluid pressure within a deformable fluid conduit, comprising:
an ultrasound array having a plurality of ultrasound transducers and disposed in a spatial configuration with respect to said fluid conduit, said ultrasound array configured and placed to acquire and track a position and geometry of said fluid conduit within a region of interest and further configured to transmit and/or receive ultrasound energy into and/or out of said fluid conduit;
a transducer driving circuit electrically coupled to said ultrasound transducers of said array so as to drive each transmitting transducer with an electrical driving signal;
a processor electrically coupled to said ultrasound transducers of said array so as to receive an electrical response signal from each receiving transducer;
a digital storage unit that stores data and program instructions allowing control of said electrical driving signals and processing said electrical response signals;
said processor further having circuitry to receive said electrical response signals and to determine at least geometric data regarding said fluid conduit, including data regarding deformation of said fluid conduit, and to determine an axial strain in said fluid conduit as well as to execute said stored program instructions and to process said stored data, including processing said program instructions, geometric data and axial strain to estimate a fluid pressure within said fluid conduit;
said processor further coupled to an output unit that conveys an output representative of said fluid pressure parameter.
